(19) United States
(12) Patent Application Publication
(10) Pub. No.: US 2007/0280889 A1
(43) Pub. Date: Dec. 6, 2007

(54) CONTRAST AGENT SYSTEM FOR X-RAY IMAGING

(76) Inventors: FRIEDRICH FUCHS, Rottenbach (DE); Georg Nollert, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 11/750,402

(22) Filed: May 18, 2007

(30) Foreign Application Priority Data
May 18, 2006 (DE) 10 2006 023 456.1

(51) Int. Cl. A61K 49/04; A61K 39/395

(52) U.S. Cl. 424/9.411; 424/178.1

(57) ABSTRACT

A contrast agent system for X-ray and computed tomography diagnostics has a dissolvable microcapsule in which a substance or substance mixture that releases CO₂ upon contact with a bodily fluid is enclosed.

### BACKGROUND OF THE INVENTION

1. Field of the Invention

The present invention concerns a contrast agent for X-ray diagnostics, such as radiographic imaging and computed tomography.

2. Description of the Prior Art

X-ray-positive substances (such as, for instance, iodine or barium compounds) are primarily used as a contrast agent for imaging with X-rays. Such contrast agents (in particular iodine compounds) can cause side effects, such as allergic reactions and impairments of the kidney function. By contrast, CO₂ causes no problems in this regard. In the form of finely distributed small bubbles, it is an X-ray-negative contrast agent, by being more permeable to X-ray radiation than body tissue. It can be applied arterially (angiography) since it is quickly resorbed and does not, like air or nitrogen, lead to embolisms. Due to the fast resorption of CO₂, the contrast-increasing effect decreases rapidly with increasing distance from the injection point. Transport, for instance to internal organs, is therefore hardly possible. A further disadvantage is that special injectors are required for application of CO₂.

### SUMMARY OF THE INVENTION

An object of the present invention to provide a contrast agent based on the release of CO₂ that can be applied in a simple manner and that offers a greater application spectrum than is the case for a conventional CO₂ injection.

This object is achieved in accordance with the invention by a contrast agent system having a substance or substance mixture that releases CO₂ upon contact with body fluid, which is enclosed in a microcapsule with a dissolving casing.

### DESCRIPTION OF THE PREFERRED EMBODIMENTS

Dissolvable microcapsules can be produced with modern techniques on the order of some micrometers and can even pass the smallest venules without the danger of a blockage or destruction. In accordance with the invention, such a microcapsule contains a substance or a substance mixture that releases CO₂ upon contrast with a body fluid. The duration up to the CO₂ release can be controlled by selection of the casing material, such that organs far removed from the application location can be reached. It is also for the casing material to be selected that concentrates in the tissue to be examined, such that this tissue can be clearly differentiated (primarily in a CT examination) from neighboring tissues after release of the carbon dioxide. In a preferred embodiment, such a tissue specificity is achieved by coupling at least one microcapsule to tissue-specific antibodies. The parenterally-administered contrast agent is initially transported to the target tissue and there couples to corresponding antigens. After resolution or resorption of the capsule casing, the capsule content comes in contact with blood or a different bodily fluid, whereby the CO₂ release is initiated. It is also possible for the substances of a substance mixture to be respectively enclosed by separate microcapsules. This variant could be advantageous when a common encapsulation of the substances poses problems, for example in production engineering.

Advantageously contained in the capsule is a substance mixture composed of an acid and a carbonate which dissolve into aqueous bodily fluid in the manner of an effervescent powder, with CO₂ generation. A gas bubble greater by the factor 10³ arises from a capsule only a few micrometers in size. A voxel size (at minimum 0.4³ mm³) that can be shown in CT is thereby achieved. If a conventional contrast agent were to be encased with a capsule, this would have to be fashioned correspondingly voluminous so that the cited voxel size is achieved after its dissolution. Such a capsule would, however, not be able to pass smaller vessels and would block these, such that a specific contrasting of a tissue would not be possible.

Tartaric acid and sodium bicarbonate are advantageously used as a CO₂-releasing substance mixture that is harmless in terms of toxicity. Given context with aqueous bodily fluid, disodium tartrate, water and CO₂ form. Disodium tartrate (CAS number: 6106-24-7) is a food additive (E335) that dissolves well and exhibits no harmful effects. A value of 30 mg/kg of body weight is specified by the European Union as an acceptable daily allowance (ADI). Toxic doses (LD50) are not known.

Fructose, glucose, mannose, galactose, sucrose, raffinose, polysaccharide or a mixture of these substances are, for example, considered as casing materials. The cited sugars dissolve in aqueous bodily fluid with a delay suitable for the purposes being discussed. Known techniques can be resorted to for the coupling of a capsule casing comprising the cited sugars to an antibody or recombinant antibody fragments or, respectively, antibody variants acquired through bioengineering.

Although modifications and changes may be suggested by those skilled in the art, it is the intention of the inventors to embody within the patent warranted hereon all changes and modifications as reasonably and properly come within the scope of their contribution to the art.

We claim as our invention:

1. A contrast agent system for X-ray diagnostics, comprising:
a dissolvable microcapsule having a casing formed of at least one substance selected from the group consisting of fructose, glucose, mannose, galactose, sucrose, raffinose and polysaccharide; and
a substance or substance mixture contained in said microcapsule, said substance or substance mixture releasing CO₂ upon contact with a body fluid.

2. A contrast agent system as claimed in claim 1 comprising said substance mixture, and comprising separate microcapsules respectively enclosing different substances in said substance mixture.

3. A contrast agent system as claimed in claim 2 wherein at least one of said separate microcapsules is coupled with a tissue-specific antibody.

4. A contrast agent system as claimed in claim 1 comprising said substance mixture, and wherein said substance mixture is formed from an acid and a carbonate.

5. A contrast agent system as claimed in claim 4 wherein said substance mixture is formed from tartaric acid and sodium bicarbonate.

6. A contrast agent system as claimed in claim 1 wherein said microcapsule is formed from at least one sugar.
